(19) United States
(12) Patent Application Publication
(10) Pub. No.: US 2010/0190883 A1
(43) Pub. Date: Jul. 29, 2010

(54) DENTAL MATERIAL

(75) Inventors: Stephan Neffgen, Hamburg (DE); Henrik Boettcher, Tostedt (DE)

Correspondence Address: Casimir Jones, S.C., 2275 DEMING WAY, SUITE 310, MIDDLETON, WI, 53562, US

(73) Assignee: ERNST MUHLBAUER GMBH & CO. KG, Norderfriedrichskoog (DE)

(21) Appl. No.: 12/692,288

(22) Filed: Jan. 22, 2010

(30) Foreign Application Priority Data
Jan. 26, 2009 (DE) 102009006173.8

(51) Int. Cl. A61K 6/10

(52) U.S. Cl. 523/109

(57) ABSTRACT

The invention provides a dental material comprising a resin matrix. It comprises:
a) at least 2% by weight of pigment with a refractive index of at least 2,
b) spherical particles and/or spherical hollow particles which are composed of optically homogeneous material and have a mean particle diameter d50 between 0.2 and 300 μm, said material having a refractive index which differs by at least 0.2 from the refractive index of the surrounding matrix and/or of the core of the hollow particles.

The invention relates to a dental material comprising a resin matrix, which is especially suitable as an impression material or bite registration material.

The CAD/CAM manufacture of prostheses in dentistry is known. Sirona Dental Systems GmbH, for example, sells a system under the CEREC® name which optically registers the intraoral tooth situation after the preparation and produces dental prosthesis by machining with reference to the optically scanned data. The optical registration of the objects to be scanned is generally accomplished with electromagnetic radiation in the range of visible light (380 to 750 nm) or in the near UV range.

To achieve a sufficiently accurate optical scan, the objects to be scanned are generally coated with commercially available matting agents of high refractive index, which comprise, for example, titanium dioxide. These matting agents are generally applied temporarily as powder or sprays.

Dental models, dental impressions or bite registrations are also subjected to optical scanning. To achieve a sufficiently accurate scan, it is known here to admix the impression or modelling materials with a filler with high refractive index in order to facilitate the optical scanning.

WO 02/11678 describes modelling materials comprising metal pigments smaller than 100 μm and more preferably smaller than 20 μm; these have good optical scanning results, but can cause a mirror effect specifically in the case of larger particles and/or especially in the case of the preferred platelets, which can lead to errors in a resulting image.

WO 2006/105579 describes a material for impressions with improved optical structure for imaging by means of photogrammetry, comprising macroparticles and microparticles in a ratio of 5-15:1. The macroparticles are larger than 1 μm and should have a size distribution in the range between approx. 30 and 200 μm or 100 and 200 μm in diameter. The particle size distribution at the surface may be between 30 and 40 μm, 30 or 35 to 150 μm. The particles may be homogeneous or inhomogeneous. The microparticles are smaller than 1 μm or smaller than 5 μm; they may be pigments. The macro- and microparticles may be polymer-based, metallic, or titanium dioxide, metal oxide, silicate. The microparticles are titanium dioxide.

WO 2006/108384 describes a two-component, addition-crosslinking silicone material for bite registration, comprising reinforcing and non-reinforcing fillers, wherein the presence of a metal oxide powder is said to improve optical scanning results, preferably of titanium dioxide with particle sizes less than 50, 20 or 2 μm.

WO 2008/064872 describes a process in which the optical registration of a dental impression is improved by roughening the surface thereof. The impression material used should contain 0.01 to 80% by weight of titanium dioxide.

DE 10103446 describes a high-viscosity, two-component silicone material which is suitable for automatic mixing and metering systems and comprises titanium dioxide as a reinforcing filler (BET<50 m²/g) and hollow and solid spheres as a non-reinforcing filler (BET>50 m²/g). The registerability of resulting impressions by means of optical scanning is not mentioned.

It is an object of the present invention to provide a dental material of the type mentioned at the outset, which enables reliable and exact optical scanning of models, impressions and the like produced therefrom.

The invention thus provides a dental material comprising a resin matrix, which comprises:
a) at least 2% by weight of pigment with a refractive index of at least 2,
b) spherical particles and/or spherical hollow particles which are composed of optically homogeneous material and have a mean particle diameter d50 between 0.2 and 300 μm, said material having a refractive index which differs by at least 0.2 from the refractive index of the surrounding matrix and/or of the core of the hollow particles.

First, some terms used in the context of the invention should be explained. The term “dental material” refers to any material usable for dental purposes, which can be used, for example, for dental restorations, but especially to produce dental models, dental impressions or bite registrations.

The dental material comprises a resin matrix. This is a polymer material which can cure, for example, after the impression is made. It may comprise curing agents which are known in the prior art and familiar to the person skilled in the art.

The inventive dental material is particularly suitable for optical scanning by means of electromagnetic radiation in the range of visible light or in the near UV range. Known optical scanning methods are, for example, optical triangulation (strip projection) or laser distance measurement.

Pigments are particles which are insoluble in the resin matrix and scatter and/or absorb light. They are preferably pigments for which the so-called scatter coefficient, which describes the light scattering capacity, significantly exceeds the light absorption and hence the so-called absorption coefficient. The pigments thus preferentially scatter a majority of the incident light, and are preferably light-coloured pigments or so-called white pigments. In principle, the pigments disclosed in the prior art cited above are suitable as inventive pigments. The refractive index of the pigments used is at least 1.5 and preferably at least 2, more preferably at least 2.5. The refractive index of pigments is generally known; it is tabulated and/or specified (for example by the manufacturer). To determine the refractive index of (pigment) powders, various methods can be employed, for example the immersion method by exchanging the embedding fluid, or by means of a temperature and/or wavelength variation method with an embedding agent (see also, for example, EP 0 832 636 B1, pages 13-14).

The inventive dental material further comprises spherical particles of optically homogeneous material. The spherical particles may preferably be spherical hollow particles. These spherical particles have substantially, or in substantial portions, spherical form or approximately spherical form.

The spherical particles consist of a predominantly optically homogeneous material. “Optically homogeneous” materials are understood here to mean those which do not have any relevant phase separation detectable by optical methods. Optical homogeneity is manifested in a good transparency, corresponding to a transparency of at least 50%. The transparency can be determined, for example, by means of the method specified in European Patent EP 0 832 638 B1, page 14. Suitable spherical particles are, for example, polymer microspheres, at least partly amorphous fillers which are produced by sol-gel processes and are composed of metal oxide, semi-metal oxide or mixed oxide, hollow microspheres composed of one of the aforementioned materials, glass microspheres or hollow glass microspheres. Preference is given to glass microspheres, hollow microspheres, for example hollow glass microspheres.

The spherical fillers additionally have at least one concave surface with a refractive index transition to the surrounding material which is characterized by a refractive index difference of at least 0.2. In the case of solid microspheres, the refractive index of the solid microspheres differs from that, for instance, of a cured resin matrix by at least 0.2. The refractive index of the solid microspheres is then preferably at least 0.2 greater than that of the cured resin matrix, but more preferably at least 0.5 greater.

In the case of hollow microspheres, the refractive index of the shell material differs by at least 0.2, more preferably by at least 0.5, from that of the core material. The refractive index of the shell material is preferably at least 0.2 greater, more preferably at least 0.5 greater, than that of the core material. The preferred core material is a gas.

It may also be preferred that the refractive index of the shell material of such hollow glass spheres does not differ significantly from that of the resin matrix.

The mean particle diameter d50 of the spherical particles is between 0.2 and 300 μm, preferred ranges being 0.4 to 200 μm, more preferably 1 to 100 μm, more preferably 1 to 50 μm. Preferred proportions by weight of the spherical particles composed of optically homogeneous material in the dental material are 1 to 50% by weight, preferably 2 to 35% by weight, more preferably 4 to 30% by weight. Suitable upper limits of the proportion by volume of spherical hollow particles in the dental material are 75% by volume, preferably 50% by volume, more preferably 40% by volume, more preferably 35% by volume. Suitable lower limits of the proportion by volume of spherical hollow particles in the dental material are 5% by volume, preferably 8% by volume, more preferably 12% by volume.

The invention has recognized that, in the prior art, optical scanning has inaccuracies, especially in the region of steep flanks (based on the direction of incidence of the scanning beam) of an object. Most optical scanning systems used in practice have illumination and recording systems (light source and light sensor) directly adjacent to one another. This means that there has to be sufficient light scatter or reflection essentially at right angles to the direction of incidence in order that detection by the light sensor can take place. In a multitude of situations, however, there is too low a rebound intensity of the light from the surface contour of the objects to be scanned, caused, for example, by an excessive penetration depth of the light into the surface contour and hence by absorption. The prior art already proposes reducing the penetration depth and hence absorption of the light by adding high-index pigments, for example titanium dioxide. This minimizes the penetration depth and increases the nonspecific reflection of the light close to the surface. Owing to the refraction laws, the intensity of the light reflected by pigments or fillers, however, also decreases with greater viewing angle (to the perpendicular of the surface to be scanned). This is true especially in the case of scanning of steep flanks, such that, in the prior art, the light intensity backscattered in the direction of the incident light beam is frequently so low that accurate recording and evaluation of the surface and contours is difficult especially in these regions.

The invention has recognized that the addition of spherical particles defined above in detail considerably facilitates the optical recording of three-dimensional objects, especially by means of triangulation.

When optical scans of a cured inventive dental material are produced, the images obtained have an improved homogeneity, image sharpness and trueness to detail, especially on steep flanks. In addition, there are fewer errors in the scan. The inventive materials can be scanned optically without further pretreatment; more particularly, no surface coating is required, for example powdering. The invention has also recognized that the spherical particles added also influence mechanically desirable properties such as Shore A hardness, consistency and extractability from organic mixing apparatus.

One possible explanation of advantages of the inventive dental material, which does not restrict the scope of protection, is that the spherical particles probably, by retroreflection, reflect back a high proportion of the incident light in the direction of the light source and hence ensure a sufficient intensity of reflection to the sensor which is generally in the immediate vicinity of the light source. This increases the light intensity available for optical analysis and evaluation, especially in steep flank regions of the material scanned, on which the incident light is at a large angle to the perpendicular of the scanned surface (in order words, at a very shallow angle relative to the surface). The invention allows a significantly improved image quality and higher image sharpness of a scanned inventive dental material compared to the prior art.

Since the spherical particles are also arranged in the form of projecting convex faces on the surface of the inventive dental material, this probably additionally leads to particularly good reflection and hence good optical scannability.

The refractive index of the cured resin matrix of the dental material, in the context of this application also referred to as surrounding matrix, is preferably low. It is preferably less than 1.55, more preferably less than 1.45.

Preferred proportions by weight of the pigment are 2-40% by weight, more preferred ranges being 4 to 30% by weight, 6 to 20% by weight and 6 to 15% by weight. The pigment may preferably have a refractive index of at least 2.5. Preference is given to inorganic pigments, especially inorganic white pigments, for example pigments selected from the group consisting of barium sulphate, zinc sulphide, calcium carbonate, zirconium dioxide and titanium dioxide. Titanium dioxide is particularly preferred.

In a particularly preferred embodiment, the inventive dental material comprises reinforcing fillers and/or non-reinforcing fillers, more preferably both reinforcing and non-reinforcing fillers.

Reinforcing fillers have a BET surface area of <50 m²/g, non-reinforcing fillers a BET surface area of >50 m²/g.

Suitable non-reinforcing fillers are metal salts, metal oxides, metal hydroxides, mixed metal oxides, glasses or mixtures thereof. Particularly suitable are silicon dioxide and/or silicates, for example cristobalite, quartz, diatomaceous earth, zirconium silicate, calcium silicate, clay minerals such as smectites, talc, zeolites, sodium aluminium silicate. Additionally particularly suitable are aluminium oxide or zinc oxide, and the mixed oxides thereof, titanium dioxide, barium sulphate, zinc sulphide, calcium carbonate, and also glass and/or plastic or composite powders and/or glass and/or plastic or composite beads.

The dental material contains preferably 1 to 80% by weight of non-reinforcing fillers, more preferably 10 to 80% by weight, even more preferably 30 to 70% by weight.

Suitable reinforcing fillers are finely divided metal salts, metal oxides, metal hydroxides, mixed metal oxides or mixtures thereof. Particularly suitable are finely divided silicon dioxide and/or silicate, for example wet-precipitated or fumed silicas, clay minerals, titanium dioxide, aluminium oxide or zinc oxide.

The dental material contains preferably 0.1 to 20% by weight of reinforcing fillers, more preferably 1 to 10% by weight, even more preferably 2 to 6% by weight.

The fillers may be surface-modified, for example silanized. The surface is preferably modified such that a reaction with the resin matrix can proceed.

The resin matrix of the inventive dental material may be selected from the group consisting of addition-crosslinking or metathesis-crosslinking polyethers or silicones, condensation-crosslinking silicones, aziridinopolyethers, reversible hydrocolloids, alginates and free-radically polymerizable resins. Among the silicones, preference is given to addition-crosslinking silicones, especially polydimethylsiloxanes. Free-radically polymerizable resins are preferably acrylates or methacrylates.

The inventive dental material may optionally comprise additives customary in the dental sector, for example stabilizers, dyes, aromas and fragrances.

The inventive dental material typically comprises curing agents. Suitable curing agents are known to those skilled in the art; the selection thereof depends on the resin matrix.

For the addition-crosslinking silicones, preference is given to platinum catalysts; for free-radical polymerization, preference is given to redox initiator systems comprising peroxides, amines, barbituric acid derivatives, urea derivatives or thiourea derivatives, resin-soluble metal salts such as copper acetate which are capable of a change in oxidation state, and ammonium halide. Particular preference is given to the barbiturate/copper salt/halide redox system.

The curing agents can be activated by means of light or chemically. Correspondingly, the inventive material may be present as one component or may consist of a plurality of components to be mixed with one another. The components are present in the form of powder, liquid or paste. The components are more preferably pastes.

The inventive material can thus also be formulated as a multi-component kit, especially two-component kit. For example, the two components may be a so-called base paste and catalyst paste.

The invention further provides for the use of an inventive dental material as a dental impression material or bite registration material. It can be used as a correction and preliminary impression material in dual-phase impression techniques, and as a monophase material in single-phase impression techniques. Particular preference is given to use as a bite registration material.

The invention is illustrated below with reference to working examples.

Impression materials were performed in each case on the basis of addition-crosslinking silicones with different proportions of transparent spherical fillers and white pigments. The impression materials are configured as two-component bite registration materials. The components consist of a base paste and of a catalyst paste, each of which is in a pasty consistency, and which are mixed with one another immediately before use. As a result of the initial mixing, the impression materials set to form cured elastomers.

The following commercially available components were used.

| | |
|---|---|
| DVPDMS 200 | Divinylpolydimethylsiloxane, viscosity 200 mPas |
| DVPDMS 1000 | Divinylpolydimethylsiloxane, viscosity |
| | 1000 mPas |
| Cristobalite | Ground β-cristobalite, Skiron SF 6000, from |
| | Quarzwerke Frechen, Germany |
| Fumed silica | Surface-modified fumed silica, HDKH 2000, from |
| | Wacker, Burghausen, Germany |
| TiO₂ | Titanium dioxide, AV 1071, from KRONOS |
| | INTERNATIONAL, INC., Leverkusen, Germany |
| Crosslinker | SiH-containing polydimethylsiloxane, Vernetzer |
| | 730, from Momentive Performance Materials, |
| | Leverkusen, Germany |
| Pt catalyst | Karstedt catalyst, 2% by weight of Pt in DVPDMS |
| | 1000 |
| DVTMDS | 1,3-Divinyltetramethyldisiloxane |
| Hollow glass | Hollow glass spheres of refractive index (shell |
| spheres | material) n = 1.51; mean particle size |
| | d₅₀ = 18 μm |
| Solid glass spheres | Solid glass spheres of refractive index |
| | n = 1.51; mean particle size d₅₀ < approx. 5 μm |

### Test Methods

### Refractive Index of Cured Resin Mixture

The liquid resin components of base paste and catalyst paste were each stirred with one another until homogeneity in beakers without adding the fillers. 1 part of the resin mixture of the base component is stirred with 1 part of resin mixture of the catalyst component and then transferred to the measurement prism of an Abbe refractometer (from Krüss, Hamburg, Germany). The refractometer is closed, the hardening of the resin matrix is awaited and the refractive index of the cured resin matrix is determined after 10 min and after 30 min after the start of mixing. The refractive index is reported for the α (D) line of sodium at 23° C.

### Processing Time

1 part by weight of base paste is mixed with 1 part by weight of catalyst paste was with the aid of a mixing spatula on a mixing block at 23±2° C. until homogeneity. Subsequently, the hardening was tested manually with the spatula at short intervals. The processing time was the period from the start of mixing until the time at which noticeable elasticity and resilience was evident in the material.

### Shore A

1 part by weight of base paste was mixed to homogeneity with 1 part by weight of catalyst paste with the aid of mixing spatula on a mixing block at room temperature (23° C.). The mixed material was transferred without bubbles into a cylindrical steel mould (internal diameter 45.0 mm, height 6.0 mm) and covered with a polymer film and a glass plate. The curing took place at 23° C. Subsequently, the test specimen was demoulded and analysed on a Shore A measuring instrument to EN ISO 868. To this end, the test specimen was analysed at three points and the mean of the three measurements was reported. The time, measured from the start of mixing, at which the Shore A measurement was carried out is reported in each case.

### Consistency

0.2 ml of the paste to be analysed was placed on a polyester film (thickness 0.01 mm, lying on a glass plate). A second polyester film of identical thickness was placed onto the paste sample. A glass plate (60×60×3.5 mm) was placed on and the arrangement was stressed with a load apparatus to measure the consistency to ISO 4823 with a weight of 1500 g for 5 s. The load apparatus and upper glass plate were removed, and the diameter of the circular paste spot formed was measured. The diameter is reported in millimetres (mm).

### Optical Scannability

The evacuated, air-free pastes were correspondingly transferred without bubbles in pairs into 50 ml double cartridges for dental use (MixPac System S50, 1:1). The paste was applied through a mixing cannula (MB 5.4-12D, from Sulzer MixPac, Rotkreuz, Switzerland) to a row of human teeth, and bitten upon. After curing, the bite register was removed, and the impression of tooth 36 was recorded optically with the aid of the camera, based on strip light projection (optical triangulation), of a CAD system (Cerec from Sirona Dental Systems GmbH, Bensheim, Germany). The sharpness and readability were assessed with reference to the intensity image.

### WORKING EXAMPLES

Parts reported as parts by weight in each case

### Example 1 (Comparative Example, Non-Inventive, 8% by Weight of TiO₂)

### Base Paste

20 parts of DVPDMS 200, 5.5 parts of DVPDMS 1000, 60 parts of cristobalite, 2 parts of fumed silica, 8 parts of TiO₂ and 4 parts of crosslinker were mixed with one another to complete homogeneity in a vacuum butterfly mixer. The paste was rolled twice through a laboratory three-roll mill (corundum rolls, from Exakt, Norderstedt, Germany) with the narrowest possible gap. Subsequently, the paste was evacuated at 20 mbar with stirring in the butterfly mixer for 10 min.

### Catalyst Paste

29 parts of DVPDMS 200, 60 parts of cristobalite, 1.98 parts of fumed silica, 8 parts of TiO₂, 1 part of Pt catalyst and 0.03 part of DVTMDS are mixed to complete homogeneity in a vacuum butterfly mixer. The paste was rolled twice through a laboratory three-roll mill (corundum rolls, from Exakt, Norderstedt, Germany) with the narrowest possible gap. Subsequently, the paste was evacuated at 20 mbar with stirring in the butterfly mixer for 10 min.

### Example 2 (Inventive) (8% by Weight of TiO₂/8% by Weight of Hollow Glass Spheres)

### Base Paste

310 parts of the base paste from Example 1 are stirred to homogeneity with 24.8 parts of hollow glass spheres in a laboratory cross-beam mixer and then evacuated with stirring at 20 mbar for 10 min.

### Catalyst Paste

310 parts of the catalyst paste from Example 1 are stirred to homogeneity with 24.8 parts of hollow glass spheres in a laboratory cross-beam mixer, and then evacuated with stirring at 20 mbar for 10 min.

### Example 3 (8% by Weight of TiO₂/25% by Weight of Solid Glass Spheres)

310 parts of the base paste from Example 1 are stirred to homogeneity with 78.1 parts of solid glass spheres (the proportion by volume is identical to the proportion by volume of the hollow glass spheres in Ex. 2) in a laboratory cross-beam mixer, and then evacuated with stirring at 20 mbar for 10 min.

### Catalyst Paste

310 parts of the catalyst paste from Example 1 are stirred to homogeneity with 78.1 parts of solid glass spheres (the proportion by volume is identical to the proportion by volume of the hollow glass spheres in Ex. 2) in a laboratory cross-beam mixer, and then evacuated with stirring at 20 mbar for 10 min.

### Example 4 (1% by Weight of TiO₂)

### Base Paste

20 parts of DVPDMS 200, 5.5 parts of DVPDMS 1000, 67 parts of cristobalite, 2 parts of fumed silica, 1 part of TiO₂ and 4 parts of crosslinker are mixed with one another to complete homogeneity in a vacuum butterfly mixer. The paste is rolled twice through a laboratory three-roll mill (corundum rolls, from Exakt, Norderstedt, Germany) with the narrowest possible gap. Subsequently, the paste is evacuated at 20 mbar in the butterfly mixer with stirring for 10 min.

### Catalyst Paste

29 parts of DVPDMS 200, 67 parts of cristobalite, 1.98 parts of fumed silica, 1 part of TiO₂, 1 part of Pt catalyst and 0.035 part of DVTMDS are mixed with one another to complete homogeneity in a vacuum butterfly mixer. The paste is rolled twice through a laboratory three-roll mill (corundum rolls, from Exakt, Norderstedt, Germany) with the narrowest possible gap. Subsequently, the paste is evacuated at 20 mbar in the butterfly mixer with stirring for 10 min.

### Example 5 (1% by Weight of TiO₂/8% by Weight of Hollow Glass Spheres)

### Base Paste

310 parts of the base paste from Example 4 are stirred to homogeneity with 24.8 parts of hollow glass spheres in a laboratory cross-beam mixer, and then evacuated with stirring at 20 mbar for 10 min.

### Catalyst Paste

310 parts of the catalyst paste from Example 4 are stirred to homogeneity with 24.8 parts of hollow glass spheres in a laboratory cross-beam mixer, and then evacuated with stirring at 20 mbar for 10 min.

### Example 6 (1% by Weight of TiO₂/25% by Weight of Solid Glass Spheres)

310 parts of the base paste from Example 4 are stirred to homogeneity with 78.1 parts of solid glass spheres (proportion by volume is identical to the proportion by volume of the hollow glass spheres in Ex. 2) in a laboratory cross-beam mixer, and then evacuated with stirring at 20 mbar for 10 min.

### Catalyst Paste

310 parts of the catalyst paste from Example 4 are stirred to homogeneity with 78.1 parts of solid glass spheres (proportion by volume is identical to the proportion by volume of the hollow glass spheres in Ex. 3) in a laboratory cross-beam mixer, and then evacuated with stirring at 20 mbar for 10 min.

Base paste

| | Example | | | | | |
|---|---|---|---|---|---|---|
| | 1 | 2 | 3 | 4 | 5 | 6 |
| | [%] | [%] | [%] | [%] | [%] | [%] |
| DVPDMS 200 | 20.10 | 18.61 | 16.06 | 20.10 | 18.61 | 16.05 |
| DVPDMS 1000 | 5.53 | 5.12 | 4.42 | 5.53 | 5.12 | 4.41 |
| Cristobalite | 60.30 | 55.83 | 48.16 | 67.34 | 62.35 | 53.79 |
| Fumed silica | 2.01 | 1.86 | 1.61 | 2.01 | 1.86 | 1.61 |
| TiO₂ | 8.04 | 7.44 | 6.42 | 1.01 | 0.93 | 0.81 |
| Crosslinker | 4.02 | 3.72 | 3.21 | 4.02 | 3.72 | 3.21 |
| Hollow glass spheres | 0 | 7.41 | 0 | 0 | 7.40 | 0 |
| Solid glass spheres | 0 | 0 | 20.12 | 0 | 0 | 20.12 |

Catalyst paste

| | Example | | | | | |
|---|---|---|---|---|---|---|
| | 1 | 2 | 3 | 4 | 5 | 6 |
| | [%] | [%] | [%] | [%] | [%] | [%] |
| DVPDMS 200 | 29 | 26.85 | 23.16 | 29.00 | 26.85 | 23.16 |
| Cristobalite | 60 | 55.55 | 47.92 | 67.00 | 62.04 | 53.51 |
| Fumed silica | 1.98 | 1.83 | 1.58 | 1.98 | 1.83 | 1.58 |
| TiO₂ | 8.00 | 7.40 | 6.39 | 1.00 | 0.92 | 0.80 |
| Pt catalyst | 1.00 | 0.93 | 0.80 | 1.00 | 0.93 | 0.80 |
| DVTMDS | 0.03 | 0.03 | 0.03 | 0.035 | 0.032 | 0.03 |
| Hollow glass spheres | 0 | 7.40 | 0 | 0 | 7.40 | 0 |
| Solid glass spheres | 0 | 0 | 20.12 | 0 | 0 | 20.12 |

Measurements

| | Example | | | | | |
|---|---|---|---|---|---|---|
| | 1 | 2 | 3 | 4 | 5 | 6 |
| Processing time/s | 60 | 60 | 60 | 60 | 45 | 45 |
| Refractive index of | 1.407 | 1.407 | 1.407 | 1.407 | 1.407 | 1.407 |
| the cured resin | | | | | | |
| Shore A | | | | | | |
| (10 min) | 88 | 90 | 90 | 89 | 91 | 91 |
| (1 h) | 88 | 90 | 90 | 90 | 91 | 91 |
| Consistency/mm | | | | | | |
| Base paste | 30 | 29 | 27 | 31 | 30 | 28 |
| Catalyst paste | 29 | | | | | |
| Manual extract- | good | good | good | good | good | good |
| ability from | | | | | | |
| cartridge | | | | | | |
| Optical | good | very | satisfactory | unreadable | readable | unreadable |
| scannability | readability | good | readability | | | |
| Intensity | | readability | | | | |
| image | | | | | | |
| Optical | satisfactory | very | satisfactory | unreadable | poor | unreadable |
| scannability | to good | good | | | | |
| Sharpness of the | | | | | | |
| intensity image | | | | | | |

By virtue especially of processing time, consistency, extractability from cartridge and Shore A hardness, the inventive materials of the examples are outstandingly suitable as bite registration materials.

In the case of Examples 1 and 2, the addition of the hollow glass spheres which had a refractive index difference between glass core and glass shell of approx. 0.5 achieved a noticeable improvement in the sharpness and readability of the intensity images. In the case of use of solid glass spheres in an analogous proportion by volume, which have a refractive index difference of only approx. 0.1 between glass and resin matrix, the readability, in contrast, was not improved.

At low proportions of titanium dioxide of less than 2% by weight, readability and sharpness are unsatisfactory.

1-14. (canceled)

15. Dental material comprising a resin matrix, characterized in that it comprises:
a) at least 2% by weight of pigment with a refractive index of at least 2,
b) spherical particles and/or spherical hollow particles which are composed of optically homogeneous material and have a mean particle diameter d50 between 0.2 and 300 μm, said material having a refractive index which differs by at least 0.2 from the refractive index of the surrounding matrix and/or of the core of the hollow particles.

16. Dental material according to claim 15, wherein the spherical particles have a mean particle diameter d50 between 0.4 and 200 μm.

17. Dental material according to claim 15 wherein the spherical particles have a mean particle diameter d50 between 1 and 100 μm.

18. Dental material according to claim 15 wherein the spherical particles have a mean particle diameter d50 between 1 and 50 μm.

19. Dental material according to claim 15, wherein the proportion of the spherical particles composed of optically homogeneous material is 1 to 50% by weight.

20. Dental material according to claim 15, wherein the proportion of the spherical particles composed of optically homogeneous material is 2 to 35% by weight.

21. Dental material according to claim 15, wherein the proportion of the spherical particles composed of optically homogeneous material is 4 to 30% by weight

22. Dental material according to claim 15, wherein the spherical particles are glass microspheres and/or hollow microspheres.

23. Dental material according to claim 15, wherein the spherical particles have a refractive index which differs by at least 0.5 from the refractive index of the surrounding matrix and/or of the core of the hollow particles.

24. Dental material according to claim 15, wherein the resin matrix after curing has a refractive index of less than 1.55.

25. Dental material according to claim 15, wherein the resin matrix after curing has a refractive index of less than 1.45.

26. Dental material according to claim 15, wherein the proportion of the pigment with a refractive index of at least 2 is between 2 and 40% by weight.

27. Dental material according to claim 15, wherein the proportion of the pigment with a refractive index of at least 2 is between 4 and 30% by weight.

28. Dental material according to claim 15, wherein the proportion of the pigment with a refractive index of at least 2 is between 6 and 20% by weight.

29. Dental material according to claim 15, wherein the proportion of the pigment with a refractive index of at least 2 is between 6 and 15% by weight.

30. Dental material according to claim 15, wherein the pigment has a refractive index of at least 2.5.

31. Dental material according to claim 15, wherein the pigment is an inorganic pigment.

32. Dental material according to claim 31, wherein said inorganic pigment is white pigment.

33. Dental material according to claim 31, wherein said inorganic pigment is selected from the group consisting of barium sulphate, zinc sulphide, calcium carbonate, zirconium dioxide and titanium dioxide.

34. Dental material according to claim 15, wherein said dental material additionally comprises nonreinforcing fillers with a BET surface area of <50 m²/g.

35. Dental material according to claim 34, wherein said dental material comprises nonreinforcing fillers in a proportion of 1 to 80% by weight.

36. Dental material according to claim 34, wherein said dental material comprises nonreinforcing fillers in a proportion of 10 to 80% by weight.

37. Dental material according to claim 34, wherein said dental material comprises nonreinforcing fillers in a proportion of 30 to 70% by weight.

38. Dental material according to claim 15, wherein said dental material comprises reinforcing fillers with a BET surface area of >50 m²/g.

39. Dental material according to claim 38, wherein said dental material comprises reinforcing fillers in a proportion of 0.1 to 20% by weight.

40. Dental material according to claim 38, wherein said dental material comprises reinforcing fillers in a proportion of 1 to 10% by weight.

41. Dental material according to claim 38, wherein said dental material comprises reinforcing fillers in a proportion of 2 to 6% by weight.

42. Dental material according to claim 15, wherein the resin matrix is selected from the group consisting of addition-crosslinking or metathesis-crosslinking polyethers or silicones, condensation-crosslinking silicones, aziridinopolyethers, reversible hydrocolloids, alginates and free-radically polymerizable resins.

43. Dental material according to claim 15, wherein said dental material is formulated as a two-component kit.

44. Dental material according to claim 15 for use as an impression material or bite registration material.
